(19) United States
(12) Patent Application Publication
(10) Pub. No.: US 2024/0381913 A1
(43) Pub. Date: Nov. 21, 2024

(54) PREPARATION AND UTILIZATION OF MICROBIAL-BASED EGG FLUID SUBSTITUTE AND MILK SUBSTITUTE

(71) Applicant: KOREA ADVANCED INSTITUTE OF SCIENCE AND TECHNOLOGY, Daejeon (KR)

(72) Inventors: Sang Yup LEE, Daejeon (KR); Kyeong Rok CHOI, Daejeon (KR); Da-Hee AHN, Daejeon (KR); Seok Yeong JUNG, Daejeon (KR)

(21) Appl. No.: 18/661,648

(22) Filed: May 12, 2024

(30) Foreign Application Priority Data
May 19, 2023 (KR) 10-2023-0064831

(51) Int. Cl. A23L 15/00; A23C 11/06; C12N 1/06

(52) U.S. Cl. CPC A23L 15/35; A23C 11/065; C12N 1/066

(57) ABSTRACT

A method of preparing a food composition for an egg fluid substitute or for a milk substitute is described, including a step of lysing microorganisms, and corresponding food compositions prepared by such method. The egg fluid substitute or milk substitute changes into a gel having properties similar to egg fluid when heated and exhibits excellent foaming performance, and can replace egg fluid in a wide range of food applications, including confectionery and baking. The described method enables the production of an egg fluid substitute or milk substitute in an environmentally friendly and sustainable manner, and provides a method for utilizing nutritionally valuable microorganisms as an egg fluid substitute or a milk substitute, as well as contributing to the food industry by expanding the choice of food ingredients.

### CROSS-REFERENCE TO RELATED APPLICATION

The priority under 35 USC § 119 of Korean Patent Application 10-2023-0064831 filed May 19, 2023 is hereby claimed. The disclosure of Korean Patent Application 10-2023-0064831 is hereby incorporated herein by reference, in its entirety, for all purposes.

### TECHNICAL FIELD

The present invention relates to a method of preparing a food composition for an egg fluid substitute or a food composition for a milk substitute, comprising the step of lysing microorganisms, and a food composition for an egg fluid substitute or a food composition for a milk substitute prepared by the method.

### RELATED ART

Due to rapid population growth and climate change, food shortages are becoming more and more visible, and proteins in particular are in shorter supply than carbohydrates and fats. On the other hand, eggs and milk are excellent sources of protein and other nutrients, and are important ingredients in many cuisines around the world. In particular, the properties of eggs, which can change their state into a gel when heated and form a stable foam when whisked, and milk, which is a colloid and tends to curdle or coagulate due to physicochemical changes such as pH, temperature, and salinity, are typical features of these ingredients and are important features in cooking and food manufacturing/processing.

On the other side, vegetarianism has gained prominence and the ethical issues of raising livestock have come to the fore, and the percentage of the population that is vegan is increasing worldwide. The rise in popularity of vegetarianism, coupled with food shortages, has led to attempts to produce plant-based egg and milk substitutes, and several plant-based egg and dairy products are commercially available. For example, plant-based egg substitutes and milk substitutes have been developed and marketed using mung beans, soy (and soy milk, tofu), almonds, and agar. In addition, recombinant microorganisms have been used to produce and purify recombinant proteins of proteins such as ovalbumin and lactalbumin, which are found in large amounts in eggs and milk, for use in the production of alternative eggs (or alternative egg whites), or yeast has been added to plant-based ingredients. However, the cells (or biomass) of the microorganisms themselves have not been used to produce alternative egg whites, alternative whole eggs, or alternative milk.

In addition, microorganisms are known to require the least amount of water and land and emit the least amount of carbon dioxide among animals (including land livestock and fish), plants, and microorganisms to produce the same amount of protein (Choi, K. R., et al., Microb. Biotechnol: 18-25 (2022)). In addition, microorganisms are an excellent nutritional resource, as the protein content in their cells (microbial biomass) is comparable to that of meat, and they are also rich in vitamins and other important nutritional components (FIG. 1).

In particular, several microorganisms having no auxotrophy, including Escherichia coli and archaea, have been reported to have the ability to utilize only carbohydrates that are relatively abundant in nature, such as glucose and starch, as a carbon source to form biomass that contains a balance of proteins, vitamins, and other nutrients, making microorganisms an ideal future high-protein food source.

In the past, there have been some attempts to use recombinant microorganisms to produce major proteins in eggs or milk, to apply physical/mechanical lysis methods such as sonication, French pressing, bead beating, blending, or freezing-thawing, chemical lysis methods using SDS (sodium dodecyl sulfate) or NaOH (sodium hydroxide), and biological/biochemical lysis methods using lysozymes to dissolve the cultured recombinant microorganisms and then purify the recombinant proteins, or to add some of the proteins purified from microorganisms to plant-based ingredients to enhance their nutritional content, but there are no reports on the production of alternative eggs or milk using the biomass of microorganisms itself.

With this background, the inventors of the present invention have endeavored to develop technologies and methods for producing materials that can replace egg fluid and dairy products, which are important protein resources, by utilizing microbial biomass, which is an environmentally friendly and highly nutritious food resource. As a result, the present invention was completed after confirming that the lysate of microorganisms has the characteristic of gelling like egg fluid when heated, and that the heated lysate of microorganisms or the heating product of diluted suspension of microorganism lysate has the characteristic of opaque colloid or suspension like milk or soy milk.

The information in this Related Art is intended solely to enhance the understanding of the background of the present invention and may not include information that would constitute prior arts known to one having ordinary skill in the art.

### SUMMARY OF INVENTION

It is an object of the present invention to provide a method of preparing a food composition for an egg fluid substitute, comprising the step of lysing microorganisms.

Another object of the present invention is to provide a method of preparing a food composition for a milk substitute, comprising the steps of heating microorganisms and lysing the heated microorganisms.

Another object of the present invention is to provide a method of preparing a food composition for a milk substitute, comprising the steps of lysing microorganisms, diluting the lysed microorganisms with water, and heating the diluted microorganisms.

Another object of the present invention is to provide a food composition for an egg fluid substitute or food composition for a milk substitute prepared by the above method.

To accomplish the above objectives, the present invention provides a method of preparing a food composition for an egg fluid substitute, comprising the step of lysing microorganisms.

The present invention also provides a method of preparing a food composition for a milk substitute, comprising the steps of heating the microorganisms; and lysing the heated microorganisms.

The present invention also provides a method of preparing a food composition for a milk substitute, comprising the steps of lysing microorganisms; diluting the lysed microorganisms with water; and heating the diluted microorganisms.

The present invention also provides a food composition for an egg fluid substitute or a food composition for a milk substitute prepared by the above method.

### BRIEF DESCRIPTION OF THE DRAWINGS

FIG. 1 is a graph depicting the composition of key nutrients in various animal, plant, and microbial species and the food products from which they are derived.

FIG. 2 is a schematic illustrating a hypothesized mechanism for how undissolved microbial biomass and microbial lysates change to sol and gel states, respectively, when heated.

FIG. 3 is a photograph showing that, when the biomass and lysates of microorganisms of examples are heated at 100° C. for 10 minutes, they change to sol and gel states, respectively, and retain their three-dimensional structure. In FIG. 3, (A) Escherichia coli Nissle 1917, a Gram-negative bacterium, (B) Bacillus subtilis, a Gram-positive bacterium, (C) Saccharomyces cerevisiae, a microorganism classified as a fungus (specifically, yeast), and (D) Cyberlindnera jadinii, represent the results of heating their biomass (left) and lysate (right), respectively.

FIG. 4 is a scanning electron micrograph image showing the surface state of biomass of (A) Escherichia coli Nissle 1917, a Gram-negative bacterium, (B) Bacillus subtilis, a Gram-positive bacterium, (C) Saccharomyces cerevisiae, a microorganism classified as a fungus (specifically, yeast), and (D) Cyberlindnera jadinii when heated at 100° C. for 10 minutes and then freeze-dried.

FIG. 5 is a scanning electron micrograph image showing the surface state of lysates of (A) Escherichia coli Nissle 1917, a Gram-negative bacterium, (B) Bacillus subtilis, a Gram-positive bacterium, (C) Saccharomyces cerevisiae, a microorganism classified as a fungus (specifically, yeast), and (D) Cyberlindnera jadinii when heated at 100° C. for 10 minutes and then freeze-dried.

FIG. 6 is a scanning electron micrograph showing the surface state of (A) whole egg fluid, (B) egg white fluid, and (C) egg yolk fluid when heated at 100° C. for 10 minutes and then freeze-dried.

FIG. 7A is a graph showing the average value (a) of the results of three compression tests of a gel prepared by heating a lysate of Escherichia coli Nissle 1917 at 100° C. for 1 hour and the analysis results (b to d) of each test. The gray shading indicates the standard deviation, and the vertical line indicates the fracture point observed in the analysis of each test.

FIG. 7B is a graph showing the average value (a) of the results of three compression tests of a gel prepared by heating a lysate of Bacillus subtilis at 100° C. for 1 hour and the analysis results (b to d) of each test. The gray shading indicates the standard deviation, and the vertical line indicates the fracture point observed in the analysis of each test.

FIG. 7C is a graph showing the average value (a) of the results of three compression tests of a gel prepared by heating a lysate of Cyberlindnera jadinii at 100° C. for 1 hour, and the results of the analysis of each test (b to d). The gray shading indicates the standard deviation, and the vertical line indicates the fracture point observed in the analysis of each test.

FIG. 8A is a graph showing the average value (a) of the results of three compression tests of a gel prepared by heating undiluted whole egg fluid at 100° C. for 1 hour, and the analysis results (b to d) of each test. The gray shading indicates the standard deviation, and the vertical line indicates the fracture point observed in the analysis of each test.

FIG. 8B is a graph showing the average value (a) of the results of three compression tests of a gel prepared by heating the diluted whole egg fluid diluted to 60% with distilled water at 100° C. for 1 hour, and the analysis results (b to d) of each test. The gray shading indicates the standard deviation, and the vertical line indicates the fracture point observed in the analysis of each test.

FIG. 8C is a graph showing the average value (a) of the results of three compression tests of a gel prepared by heating the whole egg fluid diluted to 40% with distilled water at 100° C. for 1 hour, and the analysis results (b to d) of each test. The gray shading indicates the standard deviation, and the vertical line indicates the fracture point observed in the analysis of each test.

FIG. 8D is a graph showing the average value (a) of the results of three compression tests of a gel prepared by heating the egg white fluid at 100° C. for 1 hour and the analysis results (b to d) of each test. The gray shading indicates the standard deviation, and the vertical line indicates the fracture point observed in the analysis of each test.

FIG. 8E is a graph showing the average value (a) of the results of three compression tests of a gel prepared by heating the egg yolk fluid at 100° C. for 1 hour and the analysis results (b to d) of each test. The gray shading indicates the standard deviation, and the vertical line indicates the fracture point observed in the analysis of each test.

FIG. 9A is a graph showing the average value (a) of the results of three compression tests of a gel prepared by adding heat-inactivated meat glue powder at a concentration of 1% (w/w) to a lysate of Escherichia coli Nissle 1917 and heating at 100° C. for 1 hour, and the analysis results (b to d) of each test. The gray shading indicates the standard deviation, and the vertical line indicates the fracture point observed in the analysis of each test.

FIG. 9B is a graph showing the average value (a) of the results of three compression tests of a gel prepared by adding meat glue powder with retained transglutaminase activity at a concentration of 1% (w/w) to a lysate of Escherichia coli Nissle 1917 and heating at 100° C. for 1 hour, and the analysis results (b to d) of each test. The gray shading indicates the standard deviation, and the vertical line indicates the fracture point observed in the analysis of each test.

FIG. 10 is a photograph showing the state of bubbles over time, which were formed by vortexing a lysate of Escherichia coli Nissle 1917, when the bubbles were static cultured at room temperature.

FIG. 11 shows a meringue dough prepared using only the lysate of Escherichia coli Nissle 1917 and sucrose and a meringue cookie baked using it.

FIG. 12 is a photograph showing that, when a concentrate of a lysate of Escherichia coli Nissle 1917 is heated at 100° C. for 10 min, it changes to a gel state and retains its three-dimensional structure.

FIG. 13 is a photograph of a colloidal solution obtained by heating the biomass of (A) Escherichia coli Nissle 1917 and (B) Bacillus subtilis, a Gram-positive bacterium, at 100° C. for 10 minutes, and lysing it.

FIG. 14 is a photograph of a colloidal solution obtained by heating a dilute lysate of (A) Escherichia coli Nissle 1917, and (B) Bacillus subtilis, a Gram-positive bacterium, at 100° C. for 10 minutes.

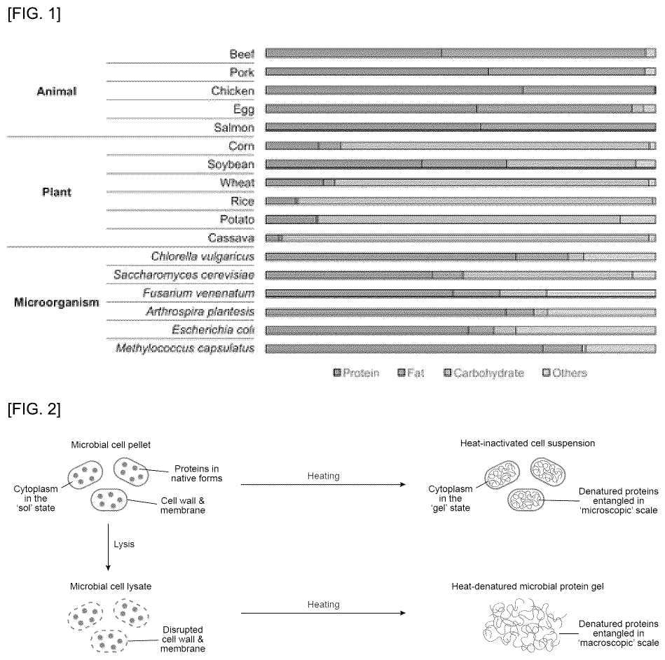

### DETAILED DESCRIPTION OF THE INVENTION AND PREFERRED EXAMPLES

Unless otherwise defined, all technical and scientific terms used herein have the same meaning as commonly understood by a person skilled in the art. In general, the nomenclature and experimental methods used herein are well known and in common use in the art.

Compared to animals (including land livestock and fish) and plants, microorganisms require less water and land to produce the same amount of protein and are known to emit the least amount of carbon dioxide (Choi, K. R., et al., Microb. Biotechnol: 18-25 (2022)).

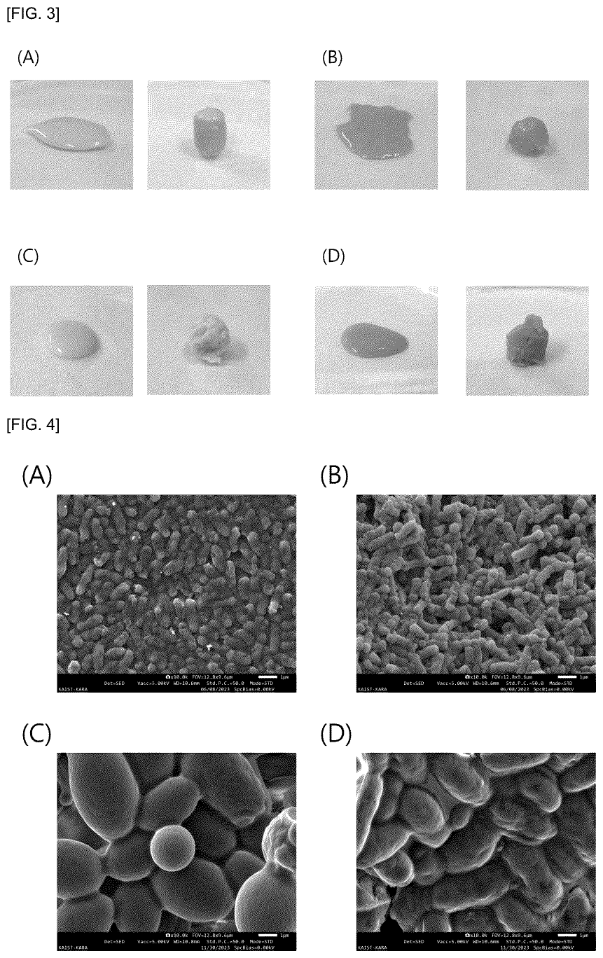

The inventors of the present invention have found, for phylogenetically diverse microorganisms, that when microorganisms with intact cellular structures are heated, due to the presence of a cell membrane or cell wall, the entanglement between proteins due to protein denaturation occurs confined to the interior of the cell, resulting in a state of sol, whereas, in the case of heating a microbial lysate obtained by destroying the cellular structure, the entanglement between proteins due to protein denaturation spreads to a wider area and a gel with properties similar to those formed by heating an egg fluid is formed (FIG. 3), and in the case of heating a concentrate of the microbial lysate, a gel is also formed (FIG. 12).

Accordingly, the present invention relates, in one aspect, to a method of preparing a food composition for an egg fluid substitute, comprising the step of lysing microorganisms.

In the present invention, the term “lysis” refers to a pretreatment method for obtaining DNA or proteins present in a cell by disrupting or damaging the cell wall and/or cell membrane of a microorganism. In the present invention, the objective of the present invention can be achieved by disrupting the cell wall or cell membrane to the extent that the cytoplasm can flow out of the cell.

A person of ordinary skill in the art will be able to utilize any lysis method known in the art to accomplish the objective of obtaining material comprising cytoplasm present inside the cell, without limitation.

For example, in the present invention, the lysis is conducted by the method selected from the group consisting of sonication, French pressing, bead beating, blending or freezing-thawing, chemical lysis methods using SDS (sodium dodecyl sulfate), chemical lysis methods using sodium hydroxide (NaOH), and biochemical lysis methods using lysozyme, biochemical lysis methods using zymolase, biochemical lysis methods using chitinase and biochemical lysis methods using β-glucanase.

The food ingredient that the present invention seeks to replace, the egg fluid, is in a colloidal state and has the physical property of gelatinizing when heated due to denaturation of the protein. In order to ensure that the lysate of microorganisms has similar physical properties to the egg fluid, it is desirable to minimize the denaturation of the proteins during the lysis of the microorganisms.

In one embodiment of the present invention, the inventors used sonication to lysate microorganisms, and to minimize protein denaturation due to the heat generated by the sonication process, the microorganisms were sonicated for a short period of time followed by sufficient cooling to minimize protein denaturation.

Thus, in the present invention, the lysis may comprise the steps of (a) treating with ultrasound for 0.1 to 3 seconds; (b) cooling for 1 to 30 seconds; and (c) repeating steps (a) and (b).

To prevent denaturation of intracellular proteins due to sonication, the sonication time in step (a) may be 4 seconds or less, 3 seconds or less, 2.5 seconds or less, preferably 2 seconds or less, more preferably 1.5 seconds or less, and most preferably 1 second or less.

For disruption of the cell wall and cell membrane by sonication, the sonication time in step (a) may be at least 0.01 seconds, at least 0.1 seconds, preferably at least 0.2 seconds, at least 0.3 seconds, more preferably at least 0.5 seconds, at least 0.8 seconds, most preferably at least 1 second.

In the present invention, the sonication time of step (a) may be 0.01 to 4 seconds, 0.1 to 3 seconds, 0.5 to 2 seconds, preferably 0.8 to 1 second.

The cooling step in step (a) is intended to remove heat generated by the sonication, and to the extent that the purpose can be accomplished, a person of ordinary skill in the art will be able to perform the cooling at a suitable temperature range and for a suitable time.

For example, the cooling of step (a) may be performed at −10 to 30° C., preferably at −5 to 15° C., more preferably at 0 to 5° C., and most preferably at 0° C.

For example, the cooling in step (a) may be performed for at least 0.1 seconds, at least 0.5 seconds, at least 0.8 seconds, preferably at least 1 second, at least 2 seconds, at least 3 seconds, at least 4 seconds, at least 5 seconds, at least 6 seconds, more preferably at least 7 seconds, most preferably at least 8 seconds, at least 9 seconds, at least 10 seconds.

For example, the cooling in step (a) is carried out for 0.1 to 60 seconds, 0.5 to 50 seconds, 0.8 to 40 seconds, preferably 1 to 30 seconds, more preferably 2 to 25 seconds, 3 to 20 seconds, most preferably 5 to 15 seconds.

To confirm that a variety of microorganisms can be utilized to prepare the food composition for an egg fluid substitute of the present invention, the inventors of the present invention have lysed various microorganisms and confirmed that the lysates can replace egg fluid. In this case, it was found that lysis occurred when yeast strains with relatively thick cell walls were sonicated for a time period of about 20 to 60 minutes, and lysis occurred when bacterial strains with relatively thin cell walls were sonicated for a time period of about 10 to 30 minutes.

In the present invention, by adjusting the total sonication time depending on the type of microorganisms to be lysed, it is possible to obtain a microbial lysate with suitable properties for the purpose of replacing the egg fluid.

In one embodiment of the present invention, the inventors have found that yeast strains, such as Saccharomyces cerevisiae and Saccharomyces cerevisiae, have egg fluid-like properties when sonication and cooling are performed repeatedly, such that the sonication time is approximately 40 minutes.

Accordingly, in the present invention, if the microorganism is yeast, step (c) may repeat the sonication so that the total sonication time of step (a) is 20 to 60 minutes.

If the microorganism is yeast, the step (c) may repeat the sonication so that the total sonication time of step (a) is 10 to 80 minutes, 20 to 60 minutes, 25 to 55 minutes, preferably 30 to 50 minutes, most preferably 35 to 45 minutes, and the person of ordinary skill in the art will be able to determine an appropriate time based on the microbial species to be lysed.

In other embodiments of the present invention, the inventors have found that bacterial strains such as Escherichia coli and Bacillus subtilis have egg-like properties when sonication and cooling are performed repeatedly, such that the sonication time is about 20 minutes.

Accordingly, in the present invention, when the microorganism is a bacterium, step (c) may repeat the sonication so that the total sonication time of step (a) is 10 to 30 minutes.

If the microorganism is a bacterium, the step (c) may repeat the sonication so that the total sonication time of step (a) is 5 to 40 minutes, 10 to 30 minutes, preferably 15 to 25 minutes, most preferably 18 to 22 minutes, and one of ordinary skill in the art will be able to determine an appropriate time based on the microbial species to be lysed without undue experimentation.

When microorganisms are lysed, the lysate contains a mixture of cytoplasm, crushed cell walls, and cell membranes and can be considered colloidal. Egg fluid is also classified as a colloid. In one embodiment of the present invention, various microorganisms were lysed, and it was confirmed that the lysate changed into a gel when heated, and the physical properties of the formed gel were checked by compression test, and it was confirmed that it has similar physical properties (Young's modulus) to the gel formed by heating the egg fluid.

Accordingly, in the present invention, the lysed microorganisms are in a colloidal state and, upon heating, turn into a gel having a young's modulus of 10 to 60 kPa.

For example, the lysed microorganisms may turn into a gel having a Young's modulus of 1 to 160 kPa, 4 to 100 kPa, preferably 7 to 80 kPa, more preferably 10 to 60 kPa, most preferably 15 to 40 kPa, 20 to 30 kPa upon heating.

In practice, compression tests have been performed on the egg fluid and it has been found that it exhibits a Young's modulus of 4 to 160 kPa, depending on the type of egg fluid and the degree of dilution. In view of the fact that the egg fluid is often diluted when used in cooking, one of ordinary skill in the art will be able to identify the properties of the lysed microorganisms described herein and take additional steps to adjust the properties of the lysed microorganisms, such as diluting or concentrating the lysed microorganisms, in order to substitute the egg fluid.

In the present invention, the microorganisms may be edible microorganisms.

In light of the purpose of using the lysed microorganisms obtained through the lysis step as an egg fluid substitute, it would be obvious to a person skilled in the art that edible microorganisms can be utilized in the method of the present invention.

For example, in the present invention, the edible microorganisms may be gram-negative bacteria, gram-positive bacteria, microalgae, yeast, mycelial fungi, and protozoa.

More specifically, for example, the edible microorganisms may be at least one selected from the group consisting of, but not limited to, Escherichia coli, Bacillus subtilis, Saccharomyces cerevisiae, Cyberlindnera jadinii, Komagataella pastoris, Komagataella phaffii, Torulopsis corallina, Geotrichum candidum, Kluyveromyces marxianus, Rhodosporidium toruloides, Yarrowia lipolytica, Aspergillus oryzae, Rhizopus oligosporus, Rhizomucor pusillus, Fusarium venenatum, Fusarium flavolapis, Trichoderma spp., Paecilomyces variotii, Monascus purpureus, Monascus ruber, Neurospora spp., Lactobacillus plantarum, Lactobacillus sakei, Rhodobacter capsulatus, Methylobacterium extorquens, Methylococcus capsulatus, Methylophilus mthylotrophus, Xanthobacter spp., Cupriavidus necator, Arthrospira spp., Chlorella spp., Dunaliella spp., Haematococcus spp. and Schizochytrium spp., and may be Chlorella spp., Spirulina (Arthrospira platensis, Arthrospira maxima, Arthrospira vulgaris), or Aphanizomenon flos-aquae, and microorganisms included in the lists of food ingredients provided by the Korea food and Drug Administration, the International Dairy Federation, the U.S. Food and Drug Administration, and the European food Safety authority can be utilized in the present invention without limitation, and are incorporated herein by reference.

Most edible microorganisms have the ability to produce abundant protein with little water and nutrients, so in view of the nutritional perspective, they have the potential to be utilized as a protein source to replace egg fluid.

To achieve their purpose as a protein source, the microorganisms of the present invention may have a crude protein content of at least 30% by weight, preferably at least 40% by weight, at least 50% by weight, more preferably at least 60% by weight, and most preferably at least 70% by weight, based on their dry weight.

In one embodiment of the present invention, by adding a food additive to the lysed microorganisms, it was found that the Young's modulus of the gel formed after heating is increased, confirming that the properties of the lysed microorganisms can be adjusted to resemble egg fluid using the food additive.

Accordingly, the present invention may further comprise the step of adding a food additive to the lysed microorganisms.

The food additives may contain various enzymes, nutrients, vitamins, minerals (electrolytes), flavoring agents such as synthetic and natural flavoring agents, coloring agents and intermediates (cheese, chocolate, etc.), pectic acid and its salts, alginic acid and its salts, organic acids, protective colloidal thickeners, pH regulators, stabilizers, preservatives, glycerin, alcohol, carbonation agents used in carbonated beverages, etc. In some cases, they may also contain natural fruit juices and pulp for the preparation of fruit juice beverages and vegetable beverages. These ingredients may be used independently or in combination. The proportions of these additives are not critical, but are typically selected in the range of 0 to about 20 parts by weight per 100 parts by weight of the composition of the present invention.

In particular, in one embodiment of the present invention, it was found that the addition of maltodextrin and/or transglutaminase increases the elasticity (Young's modulus) of the gel formed upon heating.

Accordingly, in the present invention, the food additive may be is maltodextrin and/or transglutaminase.

An egg is composed of an outer shell and an inner fluid, which is composed of egg white and yolk. In addition, the egg fluid that is a mixture of egg white and egg yolk is commonly referred to as whole egg fluid.

The lysed microorganisms prepared according to the method of the present invention can be used in the preparation of food products as a substitute for whole egg fluids, egg whites or egg yolks.

Thus, in the present invention, the egg fluid may be whole egg fluid, egg white or egg yolk.

The food composition of the present invention is rich in protein and has similar physical properties to egg fluid, and can therefore be utilized as a substitute for egg fluid in cooking with eggs and in the preparation of egg products. Accordingly, the present invention provides a food product utilizing the food composition for an egg fluid substitute prepared according to the method described above.

The food composition for an egg fluid substitute of the present invention can replace all or part of the egg fluid in the preparation of a food product. Since the present invention utilizes microorganisms in place of egg fluid, the food product may be an egg-free food product.

Based on the description herein, one of ordinary skill in the art will be able to utilize the food composition for an egg fluid substitute of the present invention in foods where egg fluid is utilized. Food products utilizing egg fluid include a broad classification of food products, such as dishes such as boiled eggs, fried eggs, scrambled eggs, egg omelets and steamed eggs; various fried foods cooked with eggs in batter; confectionery and baked goods cooked with egg fluid in baking dough; and processed food products such as mayonnaise and ice cream.

In particular, the food composition for an egg fluid substitute prepared by the method of the present invention exhibited excellent foaming performance, and it was confirmed that meringue cookies could be prepared in one embodiment of the present invention. Therefore, the present invention can be used in particular as a substitute for egg fluid in the manufacture of confectionery and baked goods.

For example, the food composition of the present invention can be utilized in the preparation of a wide range of confectionery and baked goods, such as cookies, macaroons, muffins, sponge cakes, pound cakes, chiffon cakes, pancakes, rolls, castellas, donuts, bagels, madeleines, biscuits, waffles, souffles, pies, cookie breads, cooked breads, white breads, croissants, and the like.

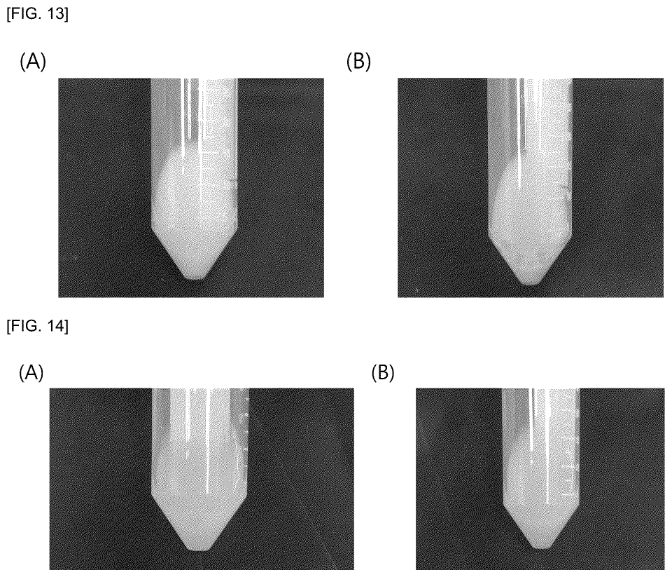

In one embodiment of the present invention, it was found that for a phylogenetically diverse range of microorganisms, when microorganisms with intact cellular structures are heated and then lysed (FIG. 13) or when diluted suspensions of microorganisms are lysed and then heated (FIG. 14), the suspensions become opaque due to protein denaturation and exhibit colloidal or suspension characteristics, exhibiting milk or soy milk-like physical properties.

The results of these tests indicate that it is possible to produce alternative milks with any type of microorganism, either by lysing the microorganisms heated in their intact state or by heating a dilute suspension of the microorganisms.

Accordingly, in another aspect, the present invention relates to a method of preparing a food composition for a milk substitute, comprising the steps of heating microorganisms; and lysing the heated microorganisms.

The above heating step denatures the protein, and only inside the undissolved microbial cell, the protein becomes entangled due to denaturation (FIG. 2), forming a sol and changing to a colloidal or suspension state. Denaturation of proteins is generally known to occur at temperatures about 55° C. or more.

Accordingly, in the present invention, the heating is carried out at 55 to 130° C., 60 to 120° C., preferably at 70 to 110° C., more preferably at 80 to 105° C., most preferably at 90 to 100° C.

The description of the lysis method and microorganisms for food composition for an egg fluid substitute described above is equally applicable to the lysis method and microorganisms for food composition for a milk substitute.

Accordingly, the lysis may be performed via a method selected from the group consisting of sonication, French pressing, bead beating, blending or freezing-thawing, chemical lysis methods using SDS (sodium dodecyl sulfate), chemical lysis methods using sodium hydroxide (NaOH), and biochemical lysis methods using lysozyme, biochemical lysis methods using zymolase, biochemical lysis methods using chitinase and biochemical lysis methods using β-glucanase.

In another embodiment of the present invention, it was found that the lysed microorganisms, even when diluted with water and then heated, change to a milk-like sol state and have physical properties similar to milk.

Accordingly, in another aspect, the present invention relates to a method of preparing a food composition for a milk substitute, comprising the steps of lysing microorganisms; diluting the lysed microorganisms with water; and heating the diluted microorganism.

The description of the lysis method and microorganisms for food composition for an egg fluid substitute described above is equally applicable to the lysis method and microorganisms for food composition for a milk substitute.

Accordingly, the lysis may be performed via a method selected from the group consisting of sonication, French pressing, bead beating, blending or freezing-thawing, chemical lysis methods using SDS (sodium dodecyl sulfate), chemical lysis methods using sodium hydroxide (NaOH), and biochemical lysis methods using lysozyme, biochemical lysis methods using zymolase, biochemical lysis methods using chitinase and biochemical lysis methods using β-glucanase.

In the present invention, the heating is carried out at 55 to 130° C., 60 to 120° C., preferably at 70 to 110° C., more preferably at 80 to 105° C., most preferably at 90 to 100° C.

In addition, edible microorganisms can be utilized for the preparation of the food composition for a milk substitute of the present invention.

For example, in the present invention, the microorganism is an edible microorganism, which is selected from the group consisting of, but not limited to, Escherichia coli, Bacillus subtilis, Saccharomyces cerevisiae, Cyberlindnera jadinii, Komagataella pastoris, Komagataella phaffii, Torulopsis corallina, Geotrichum candidum, Kluyveromyces marxianus, Rhodosporidium toruloides, Yarrowia lipolytica, Aspergillus oryzae, Rhizopus oligosporus, Rhizomucor pusillus, Fusarium venenatum, Fusarium flavolapis, Trichoderma spp.), Paecilomyces variotii, Monascus purpureus, Monascus ruber, Neurospora spp., Lactobacillus plantarum, Lactobacillus sakei, Rhodobacter capsulatus, Methylobacterium extorquens, Methylococcus capsulatus, Methylophilus mthylotrophus, Xanthobacter spp.), Cupriavidus necator, Arthrospira spp., Chlorella spp., Dunaliella spp., Haematococcus spp. and Schizochytrium spp.

The present invention is described in more detail with reference to the following examples. These examples are intended solely to illustrate the present invention, and it would be apparent to one of ordinary skill in the art that the scope of the present invention is not to be construed as limited by these examples.

### Example 1: Analyzing the Nutrient Content in the Biomass of Different Microbial Strains

To analyze the nutrients contained in the biomass of Escherichia coli Nissle 1917, a Gram-negative bacterium, and Bacillus subtilis, a Gram-positive bacterium, which have been safely consumed for many years, flask cultures were performed using MR media with the same composition as shown in Table 1 below. More specifically, 5 mL MR medium was inoculated with Escherichia coli Nissle 1917 and shaking cultured at 37° C. at 200 rpm for approximately 16 hours. Then, 500 μL of the culture was subcultured into 300 mL baffled flask containing 50 mL MR medium and shaking cultured at 30° C. at 200 rpm for approximately 16 hours. The biomass was then recovered by centrifugation at 2090×g for 10 min, frozen at −80° C., and lyophilized to obtain dried biomass powder of Escherichia coli Nissle 1917.

TABLE 1
Composition of MR Medium

| Component | Concentration | |
|---|---|---|
| Citric acid | 0.8 | g/L |
| Trace metal solution | 5 | mL/L |
| KH₂PO₄ | 6.67 | g/L |
| (NH₄)₂HPO₄ | 4 | g/L |
| KOH | Adjust pH to 7.0 | |
| Glucose | 20 | g/L |
| MgSO₄ · 7H₂O | 0.8 | g/L |

To obtain B. subtilis biomass, 5 mL MR medium (composition shown in Table 1) was inoculated with B. subtilis and shaking cultured at 37° C. at 200 rpm for approximately 24 hours. Then 500 μL of the culture was subcultured into 300 mL baffled flask containing 50 mL MR medium and shaking cultured at 30° C. at 200 rpm for approximately 24 hours. The biomass was then recovered by centrifugation at 2090×g for 10 min, frozen at −80° C., and lyophilized to obtain dried biomass powder of Bacillus subtilis.

For the analysis of the moisture content in the obtained microbial biomass, the constant volume of the weighing container was first obtained by taking a constant amount of samples and using the normal pressure drying method, and then the samples were heated in a hot air dryer (JSOF-150, JSR Co., Republic of Korea) at 135° C. for 2 hours, cooled for a certain time in a desiccator, and weighed, and the lost weight was considered as the moisture content.

The content of crude protein was determined by the Kjeldahl method according to AOAC 976.05 by taking about 0.5 g of the analyzed sample, adding a selenium-based decomposition catalyst and 12 mL of sulfuric acid, heating at 420° C. for 1 hour to decompose it, and measuring it using a Kjeltec device (Kjeltec auto 2300 Analyzer, FOSS TECATOR, Höganäs, Sweden) to obtain the content of nitrogen and multiplying it by the nitrogen factor (6.25) to calculate the content of crude protein.

Crude fat content was analyzed using the Soxhlet extraction method. It was measured using an ST243 Soxtec™ (FOSS TECATOR, Höganäs, Sweden), an automated fat extractor. An exclusive cellulose thimble was filled with 1 g of sample and the opening thereof was plugged with cotton wool to be fitted with the above equipment. The crude fat analytical vessel was fitted after drying for 1 hour, weighing, and filling with 80 mL of ethyl ether. The extractor was run through the following steps: boiling water at 110° C. for 20 min, washing for 40 min, recovery for 20 min, and drying for 20 min. The analytical vessel was then removed, the remaining ether was volatilized in a fume hood, dried in a desiccator at 100° C. for 1 hour, vacuum cooled for 30 min and weighed to determine the content of crude fat in 1 g of biomass sample.

The direct incineration method was used to analyze the crude ash content. First, the constant volume of the crucible was measured, and a sample of about 2 g was taken and pre-incinerated by heating with an electric furnace, then placed in a 600° C. electric furnace (CT-DMF2, Coretech Co., Korea) to burn for 2 hours. After cooling for 40 min in a desiccator, the weight was measured to analyze the difference in weight before and after ashing incineration to calculate the amount of crude ash.

Carbohydrate content was considered to be the remainder excluding the moisture, ash, crude protein, and crude fat content.

Furthermore, the analysis of amino acid content in the biomass was based on the modified AOAC method (2005), which is based on the ninhydrin post column reaction method using ion exchange chromatography. More specifically, to analyze the content of sulfur-free amino acids except tryptophan, 0.2 g of biomass sample was placed in a decomposition tube, 10 mL of 6 N HCl was added, nitrogen gas was injected, and it was hydrolyzed at 110° C. for 24 hours. The filtrate was concentrated with a reduced pressure concentrator and then diluted to 50 mL by adding 0.2 M sodium citrate buffer, and the filtrate filtered through a 0.20 μm cellulose acetate syringe filter was used as the analytical sample. The sulfur-containing amino acids methionine and cysteine were determined by performic acid oxidation and tryptophan was determined by alkaline hydrolysis. More specific analytical conditions are given in Table 2 and Table 3 below.

TABLE 2
Conditions for analyzing the content of amino acids except tryptophan

| Items | Conditions |
|---|---|
| Instrument | Hitachi, L-8900 |
| Column | Ion exchange column (4.6 × 60 mm, #2622PH column) |
| Mobile phase | Buffer set (PH-SET KANTO) |
| Detector | UV/Vis (440, 570 nm) |
| Flow rate | Ninhydrin ; 0.30 ml/min |
| | Buffer ; 0.35 ml/min |

TABLE 3
Conditions for analyzing the content of tryptophan

| Items | Conditions |
|---|---|
| Instrument | Waters Isocratic 600 pump, 486 UV/VIS detector |
| Column | CAPCELL PAK C18 (4.6 × 250 mm) |
| Mobile phase | 0.0085 M Sodium acetate:methanol = 95:5 |
| Detector | UV (280 nm) |
| Flow rate | 1.0 mL/min |
| Injection volume | 20 μL |

The nutrient composition of Escherichia coli Nissle 1917 and Bacillus subtilis biomass was analyzed using the methods described above and found to contain 70% or more protein on a dry mass basis, as shown in Table 4 below, and was also found to be rich in various essential amino acids.

TABLE 4
Nutritional composition of Escherichia coli Nissle 1917 and
Bacillus subtilis biomass

| | Escherichia coli | |
|---|---|---|
| Component (%) | Nissle 1917 | Bacillus subtilis |
| Water | 2.83 ± 0.03 | 2.08 ± 0.01 |
| Crude protein | 72.01 ± 0.45 | 70.29 ± 0.20 |
| Crude fat | 1.86 ± 0.18 | 5.28 ± 0.26 |
| Ash | 16.68 ± 0.15 | 15.55 ± 0.20 |
| Crude carbohydrate | 6.61 ± 0.11 | 6.80 ± 0.67 |
| Aspartic acid | 6.06 ± 0.03 | 6.35 ± 0.06 |
| Threonine | 2.83 ± 0.01 | 2.56 ± 0.03 |
| Serine | 2.20 ± 0.02 | 1.97 ± 0.02 |
| Glutamic acid | 7.02 ± 0.05 | 9.89 ± 0.11 |
| Proline | 1.91 ± 0.03 | 1.51 ± 0.01 |
| Glycine | 3.15 ± 0.02 | 2.43 ± 0.01 |
| Alanine | 4.41 ± 0.01 | 4.13 ± 0.03 |
| Valine | 3.50 ± 0.01 | 4.21 ± 0.01 |
| Isoleucine | 2.70 ± 0.02 | 2.71 ± 0.03 |
| Leucine | 4.78 ± 0.03 | 9.63 ± 0.07 |
| Tyrosine | 1.81 ± 0.08 | 1.49 ± 0.04 |
| Phenylalanine | 2.42 ± 0.01 | 2.06 ± 0.01 |
| Histidine | 1.24 ± 0.01 | 1.06 ± 0.01 |
| Lysine | 4.32 ± 0.01 | 4.03 ± 0.03 |
| Arginine | 3.74 ± 0.04 | 2.64 ± 0.01 |
| Cysteine | 0.71 ± 0.01 | 0.42 ± 0.02 |
| Methionine | 1.66 ± 0.03 | 1.50 ± 0.05 |
| Tryptophan | 0.67 ± 0.03 | 0.38 ± 0.02 |

In addition, according to FoodData Central, a database of food nutrient content provided by the U.S. Department of Agriculture, and Ouedraogo et al [African Journal of Biotechnology 16, 359-365 (2017)], the biomass of the long-consumed yeasts Saccharomyces cerevisiae and Cyberlindnera jadinii (formerly known as Candida utilis) also contain large amounts of protein and essential amino acids, as shown in Table 5 below.

TABLE 5
Nutritional composition of Saccharomyces cerevisiae and
Cyberlindnera jadinii biomass

| Component (%) | Saccharomyces cerevisiae | Cyberlindnera jadinii |
|---|---|---|
| Water | 5.08 | — |
| Crude protein | 40.4 | 54.8 ± 0.12 |
| Crude fat | 7.61 | 15.12 ± 0.98 |
| Ash | 5.65 | 8.10 ± 0.18 |
| Crude carbohydrate | 41.2 | 2.80 ± 0.20 |
| Aspartic acid | 4.15 | 8.00 ± 0.72 |
| Threonine | 1.99 | 4.10 ± 0.58 |
| Serine | 1.98 | 3.60 ± 1.02 |
| Glutamic acid | 6.47 | 15.30 ± 0.66 |
| Proline | 1.65 | 2.80 ± 0.23 |
| Glycine | 1.93 | 3.80 ± 1.96 |
| Alanine | 2.32 | 6.90 ± 0.58 |
| Valine | 2.31 | 5.50 ± 0.15 |
| Isoleucine | 1.89 | 4.80 ± 0.99 |
| Leucine | 2.92 | 7.12 ± 1.64 |
| Tyrosine | 1.13 | 2.40 ± 1.20 |
| Phenylalanine | 1.75 | 4.10 ± 0.25 |
| Histidine | 0.91 | — |
| Lysine | 3.28 | 5.14 ± 0.82 |
| Arginine | 2.03 | 3.20 ± 1.12 |
| Cysteine | 0.50 | — |
| Methionine | 0.59 | 1.58 ± 2.10 |
| Tryptophan | 0.54 | 3.90 ± 0.78 |

The four microorganisms used in the examples of the present invention are readily available to those of ordinary skill in the art, and exemplary methods of obtaining them are as follows.

E. coli Nissle 1917 is the microorganism contained in Mutaflor, a probiotic product available on the Internet (www.mutaflor.com/index.html).

B. subtilis is a publicly known microbial strain and is also available through organizations such as the Korean Collection for Type Cultures (KCTC 2217), American Type Culture Collection (ATCC 33234), DSMZ (DSM 402), and NCIMB (NCIMB 10106).

S. cerevisiae is also a publicly known strain and is readily available through organizations such as the Korean Collection for Type Cultures (KCTC 7913), American Type Culture Collection (ATCC 9763), DSMZ (DSM 1333), and NRRL (NRRL Y-567).

C. jadinii is also a known strain, and is readily available through organizations such as the Korea Culture Center of Microorganisms (KCCM 50045), American Type Culture Collection (ATCC 9256), DSMZ (DSM 70167), and NRRL (NRRL Y-1084).

### Example 2: Validating a Gel Formation Upon Thermal Treatment of Escherichia Coli Nissle 1917 Biomass Lysate

To determine whether the biomass of Escherichia coli Nissle 1917, a microorganism representative of safely ingestible Gram-negative bacteria, could be utilized as an egg fluid substitute for the lysate, the biomass of Escherichia coli Nissle 1917 was first obtained by flask culture. More specifically, 5 mL MR medium (composition shown in Table 1) was inoculated with Escherichia coli Nissle 1917 and shaking cultured at 37° C. at 200 rpm for approximately 16 hours. 500 μL of the culture was then subcultured into 300 mL baffled flask containing 50 mL MR medium and shaking cultured at 30° C. at 200 rpm for approximately 16 hours. Biomass was then recovered by centrifugation at 2090×g for 10 min and frozen at 80° C. for storage.

The biomass of Escherichia coli Nissle 1917 obtained through flask culture was transferred to a 1.5-mL microcentrifuge tube and heated at 100° C. for 10 min, and it was found that the cell pellet of Escherichia coli Nissle 1917, which was in a semi-solid state, turned into a fluid as shown in the left drawing of FIG. 3, in (A) thereof. To further confirm the hypothesis disclosed in FIG. 2, a lysate of Escherichia coli Nissle 1917 biomass was then prepared by sonication. More specifically, pellets of Escherichia coli Nissle 1917 were transferred to 50-ml conical tube, soaked in ice water for cooling, and sonicated using a Vibra-Cell VCX 750 (Sonics & Materials, Inc., USA) equipped with a 13-mm diameter extender probe. For effective cooling, sonication was performed for 1 second, followed by cooling for 9 seconds, and sonication was performed for a total of 20 min based on the sonication time. The lysate of E. coli Nissle 1917 biomass obtained by the above method was transferred to a 1.5-mL centrifuge tube and heated at 100° C. for 10 min, and it was found that the lysate, which was in a fluid state, changed into a gel form, as hypothesized in FIG. 2 (right drawing in FIG. 3, in (A) thereof).

### Example 3: Validating a Gel Formation Upon Thermal Treatment of Bacillus Subtilis Biomass Lysate

To determine if the biomass of Bacillus subtilis, a microorganism representative of safely ingestible Gram-positive bacteria, could be utilized as an egg fluid substitute for the lysate, the biomass of Bacillus subtilis was obtained by flask culture. More specifically, 5 mL MR medium (composition shown in Table 1) was inoculated with Bacillus subtilis and shaking cultured at 37° C. at 200 rpm for approximately 24 hours. Then 500 μL of the culture was subcultured into 300 mL baffled flask containing 50 mL MR medium and shaking cultured at 30° C. at 200 rpm for approximately 24 hours. Biomass was then recovered by centrifugation at 2090×g for 10 min and frozen at −80° C. for storage.

The biomass of Bacillus subtilis obtained from the flask culture was transferred to a 1.5-mL centrifuge tube and heated at 100° C. for 10 min, and it was found that the cell pellet of Bacillus subtilis, which was in a semi-solid state, turned into a fluid as shown in the left drawing of FIG. 3, in (B) thereof, like the case of Escherichia coli Nissle 1917 disclosed in Example 2. Then, to further confirm the hypothesis disclosed in FIG. 2, a lysate of B. subtilis biomass was prepared by sonication. More specifically, pellets of Bacillus subtilis were transferred to 50-ml conical tubes, soaked in ice water for cooling, and sonicated using a Vibra-Cell VCX 750 (Sonics & Materials, Inc., USA) equipped with a 13 mm diameter extender probe. For effective cooling, sonication was performed for 1 second, followed by cooling for 9 seconds, and sonication was performed for a total of 20 min based on the sonication time. The lysate of Bacillus subtilis biomass obtained by the above method was transferred to a 1.5-mL centrifuge tube and heated at 100° C. for 10 min, and it was found that the lysate, which was in a fluid state, changed into a gel form, as hypothesized in FIG. 2 (right drawing in FIG. 3, in (B) thereof).

### Example 4: Validating a Gel Formation Upon Thermal Treatment of Saccharomyces Cerevisiae Biomass Lysate

To determine if the biomass of Saccharomyces cerevisiae, the most representative microorganism (yeast) of the fungi family that is routinely consumed worldwide, could be utilized as an egg fluid substitute for the lysate, the biomass of Saccharomyces cerevisiae was obtained by flask culture using the modified YM medium disclosed in Table 6 below. More specifically, 5 mL modified YM medium was inoculated with Saccharomyces cerevisiae and shaking cultured at 30° C. at 200 rpm for approximately 24 hours. Then 500 μL of the culture was subcultured into a 300 mL baffled flask containing 50 mL MR medium and shaking cultured at 30° C. at 200 rpm for approximately 16 hours. The biomass was then recovered by centrifugation at 2090×g for 10 min, washed twice with distilled water to remove as much CO2 as possible dissolved in the culture during incubation, and frozen at −80° C. for storage.

TABLE 6
Composition of the modified YM medium

| Ingredients | Concentration |
|---|---|
| Glucose | 20 g/L |
| Yeast extract | 3 g/L |
| Malt extract | 3 g/L |
| Peptone | 5 g/L |

The Saccharomyces cerevisiae biomass obtained from flask culture was transferred to a 1.5-mL centrifuge tube and heated at 100° C. for 10 min, and it was found that the cell pellet of Saccharomyces cerevisiae, which was in a semi-solid state, turned into a fluid as shown in the left drawing of FIG. 3, in (C) thereof, like the cases of Escherichia coli Nissle 1917 and Bacillus subtilis disclosed in Examples 2 and 3. Then, to further confirm the hypothesis disclosed in FIG. 2, a lysate of Saccharomyces cerevisiae biomass was prepared by sonication. More specifically, pellets of Saccharomyces cerevisiae were transferred to a 50-ml conical tube, soaked in ice water for cooling, and sonicated using a Vibra-Cell VCX 750 (Sonics & Materials, Inc., USA) equipped with a 13 mm diameter extender probe. For effective cooling, sonication was performed for 1 second, followed by 9 seconds of cooling, and sonication was performed for a total of 40 min based on the sonication time. The lysate of Saccharomyces cerevisiae biomass obtained by the above method was transferred to a 1.5-mL microcentrifuge tube and heated at 100° C. for 10 min, and it was found that the lysate, which was in a fluid state, changed to a gel form, as hypothesized in FIG. 2 (right drawing in FIG. 3, in (C) thereof). However, a porous gel was formed as the large amount of CO2 produced by Saccharomyces cerevisiae and dissolved in the lysate eluted and expanded during the heat treatment.

### Example 5: Validating a Gel Formation Upon Thermal Treatment of Cyberlindnera Jadinii Biomass Lysate

To determine if the biomass of C. jadinii, one of the yeasts that have been safely consumed as a single-cell protein, could be utilized as an egg fluid substitute for the lysate, the biomass of C. jadinii was obtained by flask culture. More specifically, 5 mL MR medium (composition shown in Table 1) was inoculated with Cyberlindnera jadinii and shaking cultured at 30° C. at 200 rpm for approximately 16 hours. 1 mL of the culture was then subcultured into 300 mL baffled flask containing 100 mL MR medium and shaking cultured at 30° C. at 200 rpm for 20 hours. Biomass was then recovered by centrifugation at 2090×g for 10 min and frozen at −80° C. for storage.

The biomass of Cyberlindnera jadinii obtained from the flask culture was transferred to a 1.5-mL centrifuge tube and heated at 100° C. for 10 minutes, and it was found that the cell pellet of Cyberlindnera jadinii, which was in a semi-solid state, turned into a fluid as shown in the left drawing of FIG. 3, in (D) thereof, like the cases of Escherichia coli Nissle 1917, Bacillus subtilis, and Saccharomyces cerevisiae disclosed in Examples 2, 3 and 4. To further confirm the hypothesis disclosed in FIG. 2, a lysate of Cyberlindnera jadinii biomass was then prepared by sonication. More specifically, pellets of Cyberlindnera jadinii were transferred to a 50-ml conical tube and distilled water equivalent to 5% of the pellet mass was added for smooth sonication. Furthermore, sonication was performed using a Vibra-Cell VCX 750 (Sonics & Materials, Inc., USA) equipped with a 13 mm diameter extender probe while soaked in ice water for cooling. For effective cooling, sonication was performed for 1 second, followed by 9 seconds of cooling, and sonication was performed for a total of 20 min based on the sonication time. The lysate of Cyberlindnera jadinii biomass obtained by the above method was transferred to a 1.5-mL centrifuge tube and heated at 100° C. for 10 min, and it was found that the lysate, which was in a fluid state, changed into a gel form, as hypothesized in FIG. 2 (right drawing in FIG. 3, in (D) thereof).

### Example 6: Microstructural Analysis of Heat-Treated Microbial Biomass and Lysates

To further confirm the hypothesis disclosed in FIG. 2 following Examples 2, 3, 4 and 5 above, the microstructure of the various microbial biomasses and lysates was analyzed using a scanning electron microscope. The heat-treated microbial biomass and lysates prepared by performing Example 2, Example 3, Example 4 and Example 5 were frozen at −80° C. and lyophilized. As control groups, samples prepared by transferring a mixture of egg fluid (whole egg fluid), egg white fluid, and egg yolk fluid isolated from a raw egg into 1.5-mL centrifuge tubes and heating at 100° C. for 10 min were also frozen at −80° C. and lyophilized.

The particles obtained by crumbling the lyophilized samples were then attached to carbon tapes, and the surfaces of the attached samples were coated with osmium about 4 nm thick using an HPC-1SW osmium coater (Shinkuu, Japan), and the microstructure was analyzed using a JSM-IT800 field emission scanning electron microscope (JEOL Ltd., Japan).

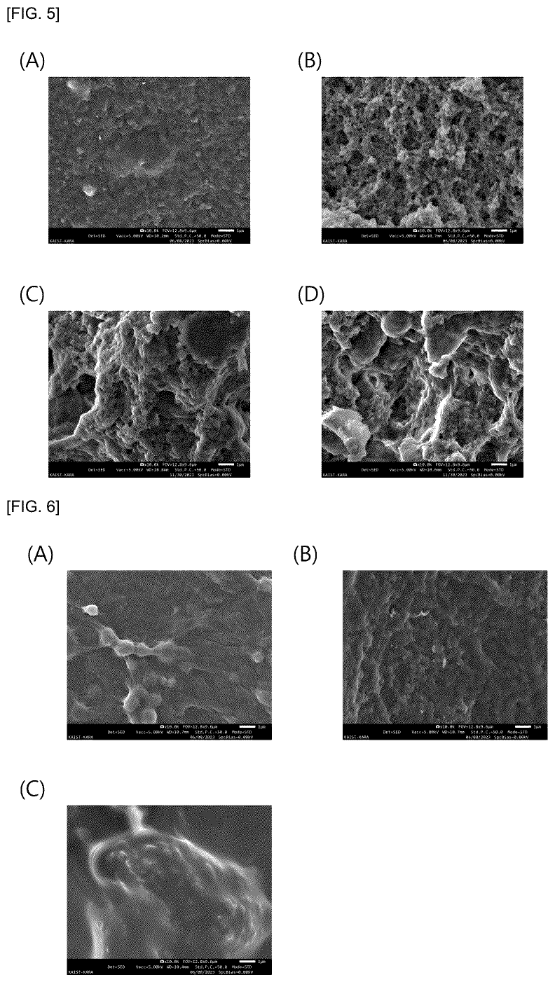

When undissolved microbial biomass was heat treated, it was found that all of Escherichia coli Nissle 1917 (FIG. 4, in (A) thereof), Bacillus subtilis (FIG. 4, in (B) thereof), Saccharomyces cerevisiae (FIG. 4, in (C) thereof), and Cyberlindnera jadinii (FIG. 4, in (D) thereof) retained intact cellular structures, as hypothesized in FIG. 2. In contrast, when the microbial lysates were heat-treated, all of Escherichia coli Nissle 1917 (FIG. 5, in (A) thereof), Bacillus subtilis (FIG. 5, in (B) thereof), Saccharomyces cerevisiae (FIG. 5, in (C) thereof), and Cyberlindnera jadinii (FIG. 5, in (D) thereof) exhibited a succession of microstructures with indistinct boundaries. These structural features were found to be similar to those of heat-treated whole egg fluid (FIG. 6, in (A) thereof), egg white fluid (FIG. 6, in (B) thereof), and egg yolk fluid (FIG. 6, in (C) thereof). These results support the hypothesis shown in FIG. 2 that lysing the microbial biomass disrupts the cell walls and membranes, allowing proteins from neighboring cells to aggregate together without physical disruption upon heating, forming a gel similar to the case of heating the egg fluid.

### Example 7: Analyzing a Gel Formed by Heat Treatment of Microbial Lysate

Compression tests were performed to analyze the properties of the gels formed by heat treatment of the microbial lysates. Lysates of Escherichia coli Nissle 1917, Bacillus subtilis, and Cyberlindnera jadinii were prepared by the methods described in Example 2, Example 3 and Example 5 above, transferred to 2-mL centrifuge tubes and heated at 100° C. for 1 hour to produce cylindrical gel samples with a diameter of 8.5 mm. The whole egg fluid obtained by mixing whole egg fluid, egg white fluid, and egg yolk fluid, and diluting the mixture to 60% or 40% with distilled water was heat treated by using the same method to prepare gel samples. The prepared samples were cut into 10 mm lengths and compression test was performed using a TXA™ multi-axis micro-texture analyzer (TXA™—Precision model, YEONJIN S-TECH, Republic of Korea) equipped with a cylindrical aluminum probe with a diameter of 35 mm and a load cell of 1-kgf, 3-kgf or 6-kgf. The aluminum probe was driven to descend at a speed of 2 mm/s, and the information of the vertical travel distance of the probe and the force exerted on the probe by the mechanical resistance of the gel were collected at a frequency of 300 point/s.

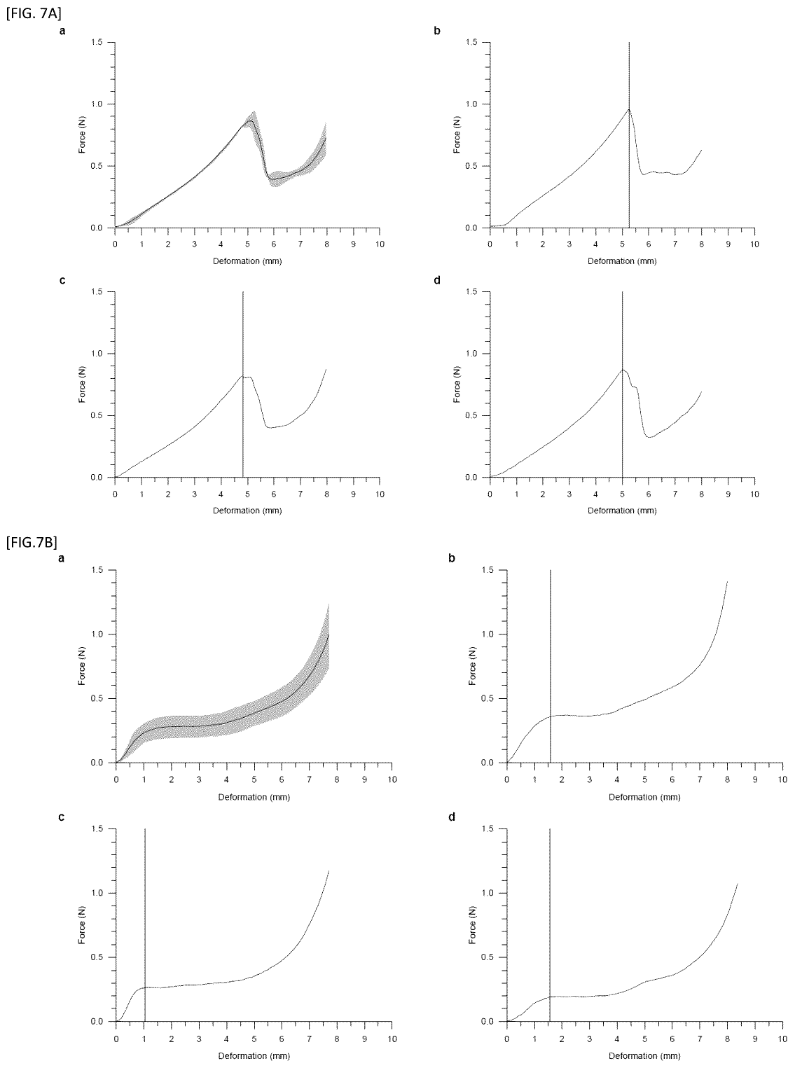

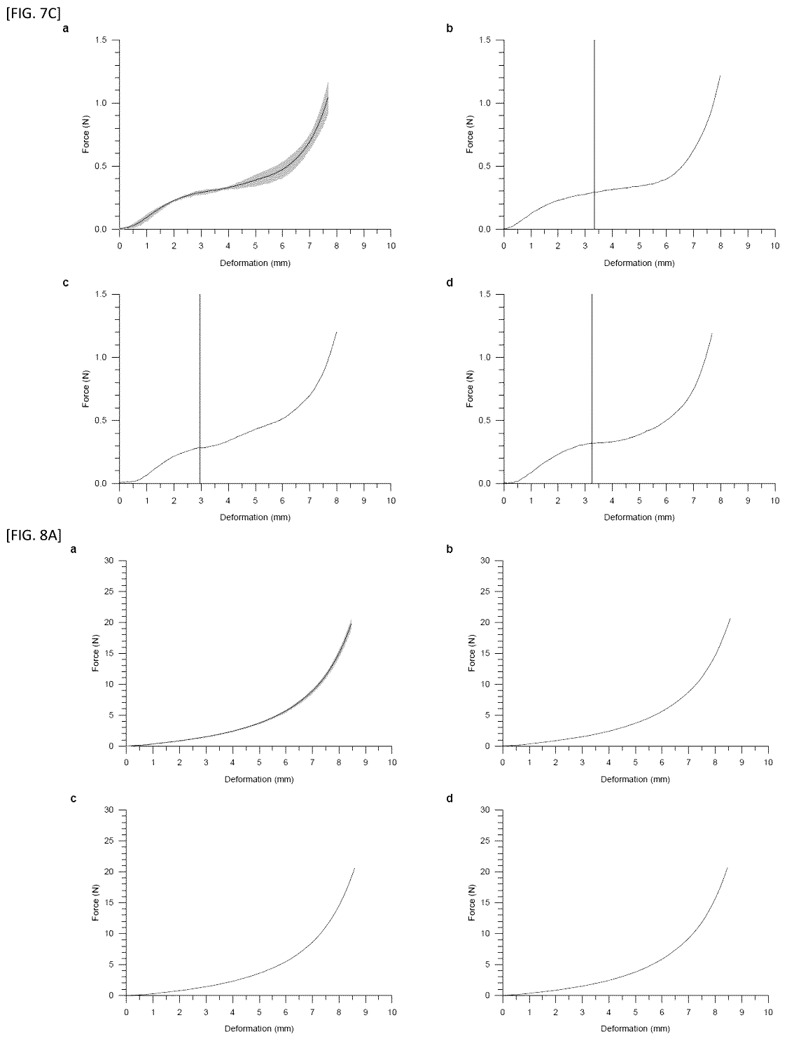

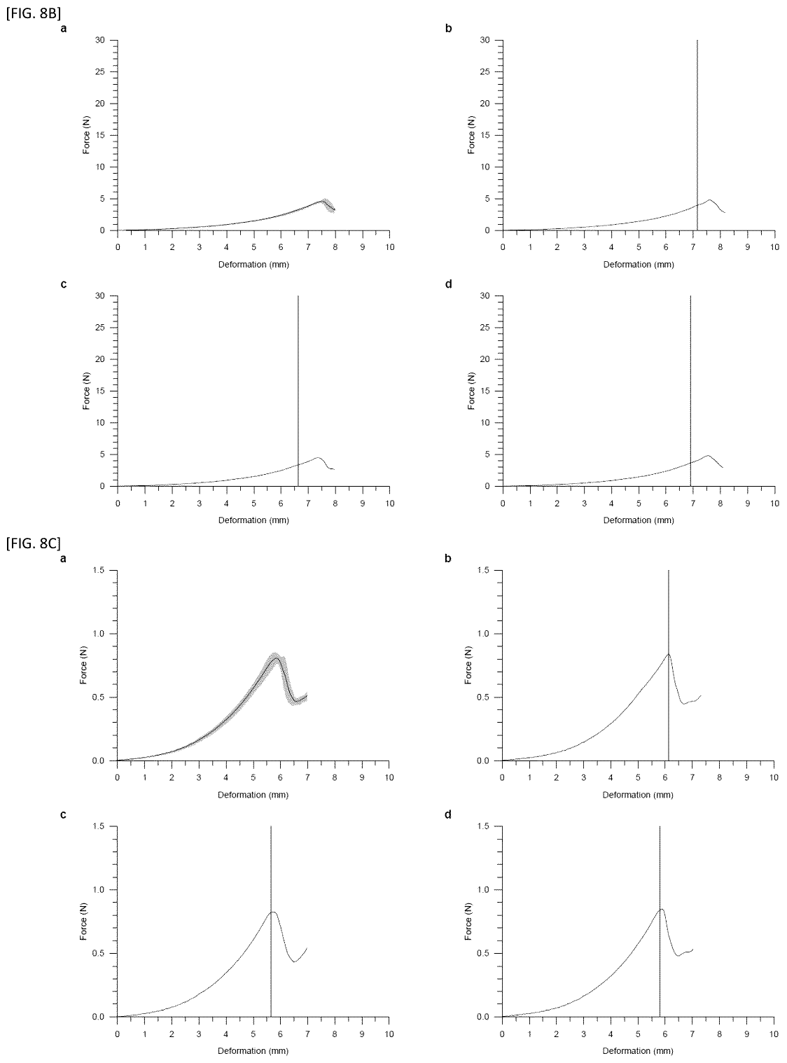

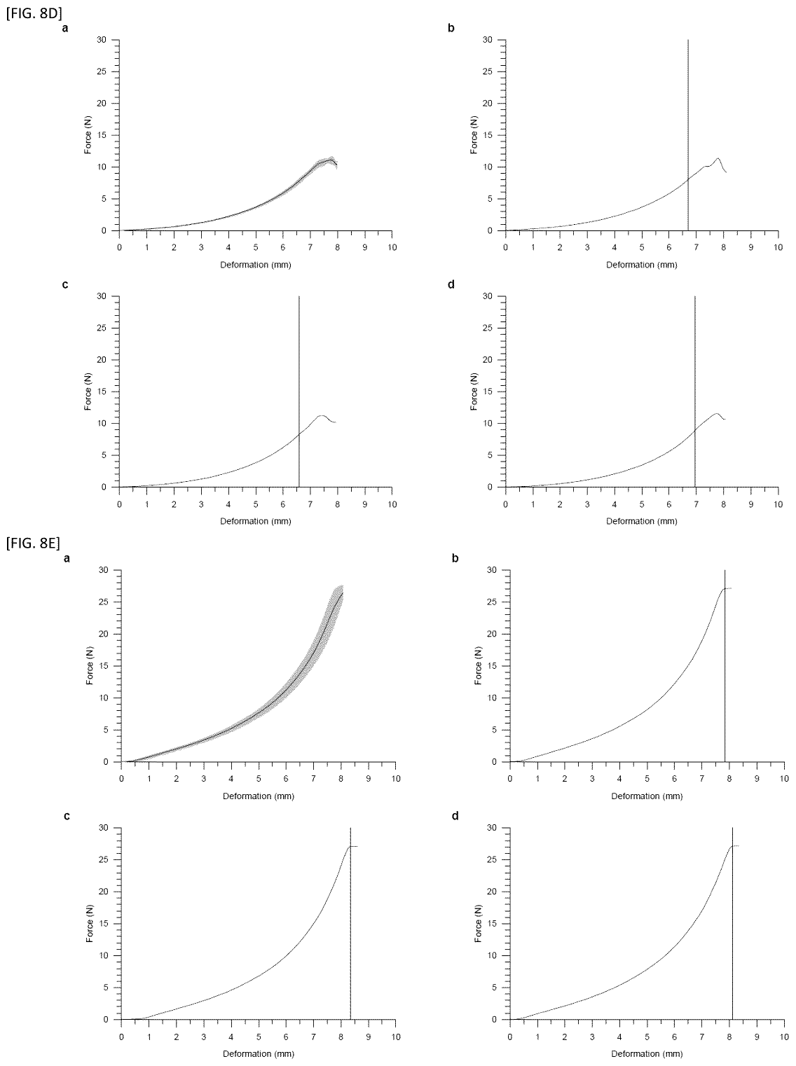

As a result, the compression profiles of a gel sample formed by heat treatment of Escherichia coli Nissle 1917 lysate (FIG. 7A), a gel sample formed by heat treatment of Bacillus subtilis lysate (FIG. 7B), a gel sample formed by heat treatment of Cyberlindnera jadinii lysate (FIG. 7C), a gel sample formed by heat treatment of undiluted whole egg fluid (FIG. 8A), a gel sample formed by heat treatment of 60% diluted whole egg fluid (FIG. 8B), a gel sample formed by heat treatment of 40% diluted whole egg fluid (FIG. 8C), a gel sample formed by heat treatment of egg white fluid (FIG. 8D), and a gel sample formed by heat treatment of egg yolk fluid (FIG. 8E) were obtained. The physical properties of the gel samples that can be determined from the compression profiles obtained above are disclosed in Table 7 below.

TABLE 7
Properties of gels formed by heat treatment of microbial lysates and various
egg fluids

| | Apparent | | Fracture | Fracture |
|---|---|---|---|---|
| Heat Treatment | Youngs | Fracture | deformation | work |
| Sample | modulus (kPa) | force (N) | (mm) | (mJ) |
| Escherichia coli Nissle | 19.3 ± 2.7 | 0.882 ± 0.070 | 5.02 ± 0.22 | 1.829 ± 0.184 |
| 1917 lysate | | | | |
| Bacillus subtilis lysate | 40.2 ± 13.4 | 0.271 ± 0.084 | 1.38 ± 0.31 | 0.211 ± 0.101 |
| Cyberlindnera jadinii | 15.9 ± 5.0 | 0.300 ± 0.019 | 3.19 ± 0.20 | 0.515 ± 0.089 |
| lysate | | | | |
| Undiluted whole egg fluid | 56.6 ± 6.0 | ND | ND | ND |
| (100%) | | | | |
| Diluted whole egg fluid | 16.6 ± 0.9 | 3.650 ± 0.299 | 6.89 ± 0.27 | 6.962 ± 0.640 |
| (60%) | | | | |
| Diluted whole egg fluid | 4.2 ± 0.3 | 0.836 ± 0.011 | 5.87 ± 0.23 | 1.442 ± 0.073 |
| (40%) | | | | |
| Egg white fluid | 37.1 ± 6.7 | 8.396 ± 0.462 | 6.75 ± 0.18 | 14.825 ± 0.729 |
| Egg yolk fluid | 125.8 ± 48.0 | 27.085 ± 0.033 | 8.11 ± 0.26 | 62.164 ± 1.625 |

*ND, not determined.

More specifically, gels formed by heat treating Escherichia coli Nissle 1917 and Cyberlindnera jadinii lysates had similar apparent Young's modulus values to gels formed by heat treating 60% diluted egg white. In addition, the gel formed by heat treating Bacillus subtilis lysate showed similar apparent Young's modulus values to the gel formed by heating the undiluted whole egg fluid and egg white fluid. In addition, the gel formed by heat treating Escherichia coli Nissle 1917 lysate showed similar fracture force, fracture deformation and fracture work values to the gel formed by heat treating 40% diluted egg white fluid. The fracture force refers to the magnitude of the force at the time of gel fracture in the compression profile, the fracture deformation refers to the compressed distance at the time of gel fracture in the compression profile, and the fracture work refers to the magnitude of the work up to the time of gel fracture in the compression profile. Based on the above results, it was confirmed that the gels formed by heat treatment of microbial lysates have similar properties to undiluted or diluted egg fluid.

### Example 8: Modulating the Properties of Microbial-Based Gels by Adding Food Compounds or Enzymes

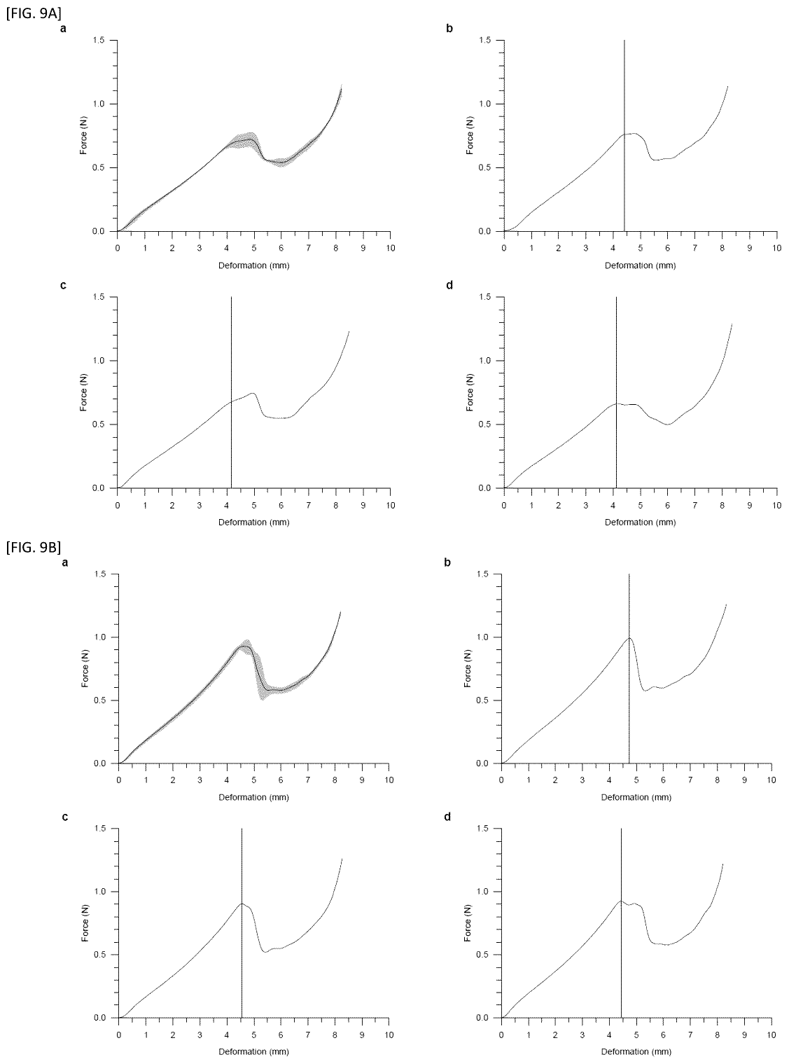

It was sought to determine whether the properties of the gel formed after heat treatment can be modulated by adding food compounds or enzymes to the microbial lysate. To this end, it was attempted to add a commercial meat glue powder (Moo Gloo TI transglutaminase, Modernist Pantry, Maine, USA) based on Streptomyces mobaraensis-derived transglutaminase, an enzyme widely used in food processing. This meat glue consists of only Streptomyces mobaraensis-derived transglutaminase and maltodextrin, and is a powdered product with only 0.02 g protein per gram of maltodextrin. Therefore, if the product is incubated at 70° C. for 4 hours to inactivate the transglutaminase, it can be considered a maltodextrin powder with very little protein. Nevertheless, when 0.2 g of heat-inactivated meat glue powder was dissolved in 20 g of Escherichia coli Nissle 1917 lysate, followed by heat treatment, a significant increase in the apparent Young's modulus value of the formed gel was observed, i.e., 19.3±2.7 kPa to 28.9±2.8 (Table 8). In addition, the values of fracture force, fracture deformation, and fracture work were analyzed and found to be significantly reduced compared to the values of gel samples prepared by heat treatment of Escherichia coli Nissle 1917 lysate without any additives (Table 8 and FIG. 9A).

TABLE 8
Properties of gels formed by heat treatment of microbial lysates added with
food compounds and enzymes

| | Apparent | | Fracture | Fracture |
|---|---|---|---|---|
| Heat Treatment | Young's | Fracture | deformation | work |
| Sample | modulus (kPa) | force (N) | (mm) | (mJ) |
| Escherichia coli Nissle | 19.3 ± 2.7 | 0.882 ± 0.070 | 5.02 ± 0.22 | 1.829 ± 0.184 |
| 1917 lysate | | | | |
| Escherichia coli Nissle | 28.9 ± 2.8 | 0.699 ± 0.054 | 4.23 ± 0.16 | 1.441 ± 0.089 |
| 1917 lysate + | | | | |
| maltodextrin (heat- | | | | |
| inactivated | | | | |
| transglutaminase) | | | | |
| Escherichia coli Nissle | 31.8 ± 2.4 | 0.940 ± 0.046 | 4.57 ± 0.15 | 1.971 ± 0.148 |
| 1917 lysate + | | | | |
| transglutaminase | | | | |

Furthermore, when 0.2 g of meat glue powder with retained enzymatic activity was dissolved in 20 g of Escherichia coli Nissle 1917 lysate and incubated at room temperature for 8 hours before heat treatment, the apparent Young's modulus value was further increased to 31.8±2.4 kPa, and the values of fracture force, fracture deformation, and fracture work were also improved compared to the addition of the heat-inactivated product (Table 8 and FIG. 9B). Based on these results, it was demonstrated that the addition of food compounds or enzymes such as maltodextrin and transglutaminase to the microbial lysate can modulate the properties of the gel formed after heat treatment.

### Example 9: Preparing Stable Foams Using Microbial Lysates

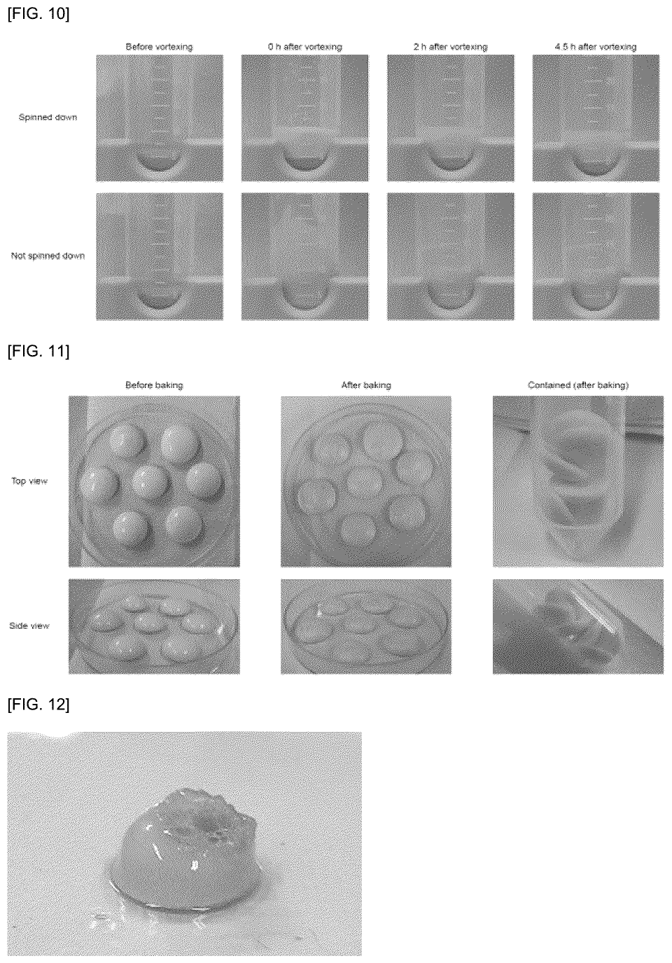

In addition to the gel formed by heating the egg fluid, the stable foam that can be made from the egg fluid is also an essential ingredient in many recipes. Therefore, it was sought to determine whether stable foam could also be prepared from microbial lysates. 5 g of Escherichia coli Nissle 1917 lysate prepared by the method described in Example 2 was transferred to a 50-ml conical tube and vortexed for 5 minutes to induce foam formation. It was found that, when subsequent spin down was performed to separate the fluid remaining after foam formation, or was static cultured at room temperature for 4.5 hours while omitting the spin down process, the formed foam was stable (FIG. 10). These results confirm that microbial lysates can form stable foams, which is an important factor in various culinary applications, such as egg fluid.

### Example 10: Preparing a Food Using Microbial Lysates

To verify whether the microbial lysate can indeed replace the egg fluid in dishes that use egg fluid as an ingredient, it was attempted to make meringue cookies using only the microbial lysate and sucrose. First, 5 g of Escherichia coli Nissle 1917 lysate prepared by the method described in Example 2 was transferred to a 50-ml conical tube and vortexed for 5 minutes to induce foam formation. Then, 5 g of sucrose was added to the Escherichia coli Nissle 1917 lysate formed with foams in three batches and whisked using a pair of disposable loops to prepare the meringue batter. Additional sucrose was added after the first addition was completely dissolved, and after all 5 g of sucrose had been added, the batter was whisked until the 50-ml conical tube was turned upside down and the meringue batter did not flow. The dough was then transferred to a squeeze bag and squeezed into the shape of meringue cookies on a plastic plate (FIG. 11). The meringue dough was then baked in a forced convection oven (OF-12GW, Lab Companion, Republic of Korea) preheated to 130° C. for 1.5 hours, and cooled at room temperature. As a result, microbial meringue cookies were baked with the same shape and texture as those made using egg fluid, as shown in FIG. 11. This example demonstrated that the microbial lysate can indeed replace the egg fluid during cooking.

### Example 11: Forming a Gel by Heat Treatment of Concentrated Microbial Lysate

The Escherichia coli Nissle 1917 lysate prepared by the method of Example 2 was lyophilized without heat treatment, and then a concentrated Escherichia coli Nissle 1917 lysate was prepared by adding a trace amount of distilled water. When this concentrated lysate was heat treated at 100° C. for 10 minutes, a gel was also formed, as shown in FIG. 12.

### Example 12: Producing a Milk Substitute Using Microbial Biomass

It was confirmed that when the culture of Escherichia coli Nissle 1917 obtained by the method of Example 2 was heat treated at 100° C. for 10 minutes and then dissolved by sonication, a liquid having the characteristics of an opaque colloid or suspension such as milk, soy milk, etc. can be prepared (FIG. 13, in (A) thereof). It was also confirmed that when the culture of Bacillus subtilis obtained by the method of Example 3 was heat treated at 100° C. for 10 minutes and then dissolved by sonication, a liquid having the characteristics of an opaque colloid or suspension, such as milk, soy milk, etc. can be prepared (FIG. 13, in (B) thereof).

Furthermore, it was confirmed that a liquid having the characteristics of an opaque colloid or suspension, such as milk, soy milk, etc., can be prepared by diluting the lysate of Escherichia coli Nissle 1917 obtained by the method of Example 2 by 5 to 10 times and heat treating it at 100° C. for 10 minutes (FIG. 14, in (A) thereof). Similarly, it was confirmed that a liquid having the characteristics of an opaque colloid or suspension, such as milk, soy milk, etc. can be prepared by diluting the lysate of Bacillus subtilis obtained by the method of Example 3 by 5 to 10 times and heat treating it at 100° C. for 10 minutes (FIG. 14, in (B) thereof).

### Industrial applicability

The egg fluid substitute produced by the method of the present invention is characterized by changing into a gel with properties similar to egg fluid when heated, and exhibits excellent foaming performance, and can therefore be used as a substitute for egg fluid in a wide range of food applications including confectionery and baking. The method of the present invention enables the production of an egg fluid substitute or a milk substitute in an environmentally friendly and sustainable manner. The present invention paves the way for a sustainable food industry by providing a method for utilizing nutritionally valuable microorganisms as an egg fluid substitute or a milk substitute. Furthermore, the utilization of the microbial lysate provided by the present invention as an egg fluid substitute or milk substitute can contribute to expanding the food industry by broadening food choices.

While the foregoing has described in detail certain aspects of the present invention, it would be apparent to one of ordinary skill in the art that these specific descriptions are merely preferred examples and are not intended to limit the scope of the present invention. Accordingly, the substantial scope of the present invention is defined by the appended claims and their equivalents.

What is claimed is:

1. A method of preparing a food composition for an egg fluid substitute, comprising the step of lysing microorganisms.

2. The method of preparing a food composition for an egg fluid substitute according to claim 1, wherein the lysis is carried out by the method selected from the group consisting of sonication, French pressing, bead beating, blending, freeze-thawing, chemical lysis using sodium dodecyl sulfate (SDS), chemical lysis using sodium hydroxide (NaOH), and
biochemical lysis using lysozyme, biochemical lysis using zymolase, biochemical lysis using chitinase, and biochemical lysis using β-glucanase.

3. The method of preparing a food composition for an egg fluid substitute according to claim 2, wherein the lysis comprises the steps of:
(a) treating with ultrasound for 0.1 to 3 seconds;
(b) cooling for 1 to 30 seconds; and
(c) repeating steps (a) and (b).

4. The method of preparing a food composition for an egg fluid substitute according to claim 3, wherein if the microorganism is yeast, step (c) repeats the sonication such that the total sonication time of step (a) is 20 to 60 minutes.

5. The method of preparing a food composition for an egg fluid substitute according to claim 3, wherein if the microorganism is a bacterium, step (c) repeats the sonication such that the total sonication time of step (a) is 10 to 30 minutes.

6. The method of preparing a food composition for an egg fluid substitute according to claim 1, wherein the lysed microorganisms of claim 1 are in a colloidal state and turn into a gel having a young's modulus of 10 to 60 kPa upon heating.

7. The method of preparing a food composition for an egg fluid substitute according to claim 1, wherein the microorganism is an edible microorganism.

8. The method of preparing a food composition for an egg fluid substitute according to claim 7, wherein the edible microorganism is a gram-negative bacterium, a gram-positive bacterium, a microalga, a yeast, a mycelial fungus, or a protozoa.

9. The method of preparing a food composition for an egg fluid substitute according to claim 7, wherein the edible microorganism is at least one selected from the group consisting of Escherichia coli, Bacillus subtilis, Saccharomyces cerevisiae, Cyberlindnera jadinii, Komagataella pastoris, Komagataella phaffii, Torulopsis corallina, Geotrichum candidum, Kluyveromyces marxianus, Rhodosporidium toruloides, Yarrowia lipolytica, Aspergillus oryzae, Rhizopus oligosporus, Rhizomucor pusillus, Fusarium venenatum, Fusarium flavolapis, Trichoderma spp., Paecilomyces variotii, Monascus purpureus, Monascus ruber, Neurospora spp., Lactobacillus plantarum, Lactobacillus sakei, Rhodobacter capsulatus, Methylobacterium extorquens, Methylococcus capsulatus, Methylophilus mthylotrophus, Xanthobacter spp., Cupriavidus necator, Arthrospira spp., Chlorella spp., Dunaliella spp., Haematococcus spp. and Schizochytrium spp.

10. The method of preparing a food composition for an egg fluid substitute according to claim 1, further comprising the step of adding a food additive to the lysed microorganisms.

11. The method of preparing a food composition for an egg fluid substitute according to claim 10, wherein the food additive is maltodextrin and/or transglutaminase.

12. The method of preparing a food composition for an egg fluid substitute according to claim 1, wherein the egg fluid is whole egg fluid, egg white or egg yolk.

13. A method of preparing a food composition for a milk substitute, comprising the steps of:
heating the microorganisms; and
lysing heated microorganisms.

14. The method of preparing a food composition for a milk substitute according to claim 13, wherein the heating is performed at 55 to 130° C.

15. The method of preparing a food composition for a milk substitute according to claim 13, wherein the lysis is carried out by the method selected from the group consisting of sonication, French pressing, bead beating, blending, freeze-thawing, chemical lysis using sodium dodecyl sulfate (SDS), chemical lysis using sodium hydroxide (NaOH), and
biochemical lysis using lysozyme, biochemical lysis using zymolase, biochemical lysis using chitinase, and biochemical lysis using β-glucanase.

16. The method of preparing a food composition for a milk substitute according to claim 13, wherein the microorganism is an edible microorganism, which is at least one selected from the group consisting of Escherichia coli, Bacillus subtilis, Saccharomyces cerevisiae, Cyberlindnera jadinii, Komagataella pastoris, Komagataella phaffii, Torulopsis corallina, Geotrichum candidum, Kluyveromyces marxianus, Rhodosporidium toruloides, Yarrowia lipolytica, Aspergillus oryzae, Rhizopus oligosporus, Rhizomucor pusillus, Fusarium venenatum, Fusarium flavolapis, Trichoderma spp., Paecilomyces variotii, Monascus purpureus, Monascus ruber, Neurospora spp., Lactobacillus plantarum, Lactobacillus sakei, Rhodobacter capsulatus, Methylobacterium extorquens, Methylococcus capsulatus, Methylophilus mthylotrophus, Xanthobacter spp., Cupriavidus necator, Arthrospira spp., Chlorella spp., Dunaliella spp., Haematococcus spp. and Schizochytrium spp.

17. A method of preparing a food composition for a milk substitute, comprising the steps:
lysing microorganisms;
diluting the lysed microorganisms with water; and
heating diluted microorganisms.

18. The method of preparing a food composition for a milk substitute according to claim 17, wherein the lysis is carried out by the method selected from the group consisting of sonication, French pressing, bead beating, blending, freeze-thawing, chemical lysis using sodium dodecyl sulfate (SDS), chemical lysis using sodium hydroxide (NaOH), and
biochemical lysis using lysozyme, biochemical lysis using zymolase, biochemical lysis using chitinase, and biochemical lysis using β-glucanase.

19. The method of preparing a food composition for a milk substitute according to claim 17, wherein the heating is performed at 55 to 130° C.

20. The method of preparing a food composition for a milk substitute according to claim 17, wherein the microorganism is an edible microorganism, which is at least one selected from the group consisting of Escherichia coli, Bacillus subtilis, Saccharomyces cerevisiae, Cyberlindnera jadinii, Komagataella pastoris, Komagataella phaffii, Torulopsis corallina, Geotrichum candidum, Kluyveromyces marxianus, Rhodosporidium toruloides, Yarrowia lipolytica, Aspergillus oryzae, Rhizopus oligosporus, Rhizomucor pusillus, Fusarium venenatum, Fusarium flavolapis, Trichoderma spp., Paecilomyces variotii, Monascus purpureus, Monascus ruber, Neurospora spp., Lactobacillus plantarum, Lactobacillus sakei, Rhodobacter capsulatus, Methylobacterium extorquens, Methylococcus capsulatus, Methylophilus mthylotrophus, Xanthobacter spp., Cupriavidus necator, Arthrospira spp., Chlorella spp., Dunaliella spp., Haematococcus spp. and Schizochytrium spp.
